(19) United States
(12) Patent Application Publication
(10) Pub. No.: US 2010/0310416 A1
(43) Pub. Date: Dec. 9, 2010

(54) METHODS AND APPARATUS FOR IMPROVED CLEANING AND DISINFECTING OF ITEMS

(76) Inventor: William J. Dalo, Santa Ana, CA (US)

Correspondence Address: KLEIN, O'NEILL & SINGH, LLP, 18200 VON KARMAN AVENUE, SUITE 725, IRVINE, CA, 92612, US

(21) Appl. No.: 12/732,142

(22) Filed: Mar. 25, 2010

Related U.S. Application Data
(60) Provisional application No. 61/163,207, filed on Mar. 25, 2009.

(51) Int. Cl. A61L 2/16; G05B 17/00

(52) U.S. Cl. 422/33; 422/116

(57) ABSTRACT

Methods and apparatus for cleaning and disinfecting items are generally discussed herein with particular discussions extended to controlled delayed release of agents for the cleaning of contact lenses.

### CROSS-REFERENCE TO RELATED APPLICATION

This is a regular utility application of provisional application Ser. No. 61/163,207 filed Mar. 25, 2009, the contents of which are expressly incorporated herein by reference.

### FIELD OF ART

Methods and apparatus for cleaning and disinfecting articles for use are generally discussed herein with particular discussion extended to timely controlled delay release of agents involved in the cleaning and disinfecting of contact lenses.

### BACKGROUND

Cleaning and disinfecting articles for use is a medical, hygienic, and sanitary practice often required and/or utilized to minimize the risk of microbial contamination or infection. A number of non-chemical as well as chemical means to bring about the cleaning and disinfecting of items are known. Non-chemical examples include heat, ultra-violet light, microwaves, and mechanical force, etc. Chemical examples include various acids and bases, aldehydes, as well as ozone, hydrogen peroxide, chlorine dioxide and various biguanides, etc.

Among the known chemical cleaners and disinfectants, hydrogen peroxide is often preferred due to its proven relative safety, the green chemistry of the byproducts. As such, hydrogen peroxide will be used herein as an exemplary chemical disinfectant. Disinfectants such as hydrogen peroxide are potent cleaners; however, they can cause irritation to the skin, the eyes, etc. Therefore, the solution needs to be neutralized after cleaning and disinfecting to minimize the irritating effect. However, if the solution is neutralized too early, its cleaning effect will diminish before the articles are appropriately cleaned. Thus, it is generally desirable to maintain the potency of the cleaning and disinfecting agent(s) over a prolonged period of time and to neutralize the disinfectants only after a desired time. This is especially true in the cleaning and disinfecting of contact lenses where it is desirable to maintain hydrogen peroxide at the initial level, over a sustained period of time so as to allow the cleaning of more resistant debris and the killing of certain more resistant microbes. As such, contact lenses will be used as an exemplary item to be disinfected. Moreover, in some instances, such as with contact lenses, it is further desirable to add the neutralizing agent concurrently with the disinfecting agent, but to delay their interactions by some means, such as by coatings on tablets, or rate controlled constructs, until some desirable benefit is obtained.

One deficiency, among others, of tablet mediated delayed release systems is that the tablet coating material(s) become tacky during dissolution. This compromises the safe and effective use of such systems since, if the container is not maintained upright until tablet dissolution is complete, the tablet can stick to the bottom of the container where it may not be in liquid solution and consequently does not bring about neutralization. Another deficiency of such systems is that after the neutralization, the agent(s) comprising the tablet remain dissolved in the neutralized solution and are available to support microbe growth. Yet another deficiency of such systems is that consistent and reproducible coating is in practice difficult to achieve and such systems are known to be highly variable in delayed release rates. A further deficiency of such systems is that it has been found necessary to include an indicator into the tablets to indicate that the tablet has indeed been added to bring about the neutralization. Again a further deficiency of such systems is that optimal tonicity in the final neutralized solution is, in practice, difficult to achieve since it requires the user to use an exact amount of liquid, not doing so results in variable tonicities, which can irritate the eye upon lens installation. A yet further deficiency of such systems is residue formed from incomplete dissolution of tablet components or non-homogeneous regions in the liquid created by density or other such differences between tablet components and bulk liquid. In view of the foregoing problems, alternative delayed release systems have been proposed and pursued.

Another alternative delayed release system uses mechanical constructs or apparatus. In addition to the above-mentioned tablet mediated release deficiencies, controlled release apparatus or constructs, while in theory may be made to work, in practice have proved technically difficult to be consistently functional.

Other alternative systems, while not delayed systems, rely on insoluble solid catalytic neutralizing agents, such as metals, metals deposited onto supports, or catalase bonded to supports, to neutralize the disinfectant.

A deficiency of both tablet based systems and alternate construct systems is that they are particular to orientation for effectively venting of the generated gas. As the vast majority of these systems use hydrogen peroxide as the disinfectant and its neutralization generates a substantial quantity of gaseous oxygen, such systems require ventilation of the gas from the contact lens container to avoid cracking, breaking, or shattering of the container. One deficiency of the existing venting systems is that they allow liquid to leak under normally anticipated conditions of usage. Another deficiency of both tablet based systems and alternate construct systems is that they are particular to orientation to function effectively.

Thus, there remains a need for more robust controlled delayed systems for cleaning and disinfecting items.

### SUMMARY

The present invention meets the aforementioned needs by providing methods and apparatus for controlled delayed release of neutralizing agents for the cleaning and disinfecting of articles for use, such as contact lenses. By sizing the apparatus appropriately, other articles such as scalpels, scissors, and other medical instruments may be cleaned and disinfected.

The present invention may be practiced by providing an apparatus adapted to delay the release of a primary agent, the apparatus comprising: a first chamber finable with a disinfectant agent and comprising a housing comprising a side wall, a top wall, a projection extending away from an internal housing wall and having a hollow bore, a secondary agent disposed, at least in part, in the hollow bore of the projection; wherein the primary agent being removably secured to the projection and covers an opening in the internal cap wall; a second chamber comprising a housing of larger volume than the first chamber having multiple vent holes and in contact with the projection; wherein the primary agent is displaceable from the projection by pressure generated in the first chamber after a time period measured from when the housing of the first chamber is coupled to the housing of the second chamber.

The present invention may also be practiced by providing an apparatus for the timely controllable cleaning and disinfecting of articles for use, the apparatus comprising: a first chamber having a first volume; a second chamber having a second volume; a seal isolating the first chamber from the second chamber; a stem projecting from the first chamber into the second chamber; and wherein the seal is severable by pressure build up in the first chamber; and wherein a channel is created and linking the first chamber with the second chamber when the seal is severed.

In yet another aspect of the present invention, there is provided a method for delaying the release of a primary agent during the cleaning and disinfecting of articles for use. The method comprising: adding a disinfectant agent into a container to a filled level; placing an article to be disinfected into a holder; placing a cap over the container and so that at least a portion of the holder is below the filled level of the disinfectant agent; maintaining a neutralizing agent below the cap but above the filled level of the disinfecting agent; moving the neutralizing agent into the disinfectant agent after a period of time in which pressure build up inside the cap and container is greater than atmospheric pressure.

Other aspects and variations of the apparatus and methods summarized above are also contemplated and will be more fully understood when considered with respect to the following disclosure.

### BRIEF DESCRIPTION OF THE DRAWINGS

These and other features and advantages of the present invention will become appreciated as the same become better understood with reference to the specification, claims and appended drawings wherein:

FIG. 1 is an exemplary exploded perspective view of a cleaning/disinfecting assembly provided in accordance with aspects of the present invention;

FIG. 2 is an isometric view of the cleaning/disinfecting assembly of FIG. 1 in an assembled state;

FIG. 3 is an exemplary top view of the assembly of FIG. 2;

FIG. 4 is an illustration of a cross-sectional side view of the assembly of FIG. 3 taken along line A-A; and

FIG. 5 is a pressure versus volume of Hydrogen peroxide versus headspace within the assembly graph.

### DETAILED DESCRIPTION

The detailed description set forth below in connection with the appended drawings is intended as a description of the presently preferred embodiments of the apparatus and method provided in accordance with aspects of the present invention and is not intended to represent the only forms in which the present invention may be constructed or utilized. The description sets forth the features and the steps for constructing and using the apparatus of the present invention in connection with the illustrated embodiments. It is to be understood, however, that the same or equivalent functions and structures may be accomplished by different embodiments that are also intended to be encompassed within the spirit and scope of the invention. As denoted elsewhere herein, like element numbers are intended to indicate like or similar elements or features.

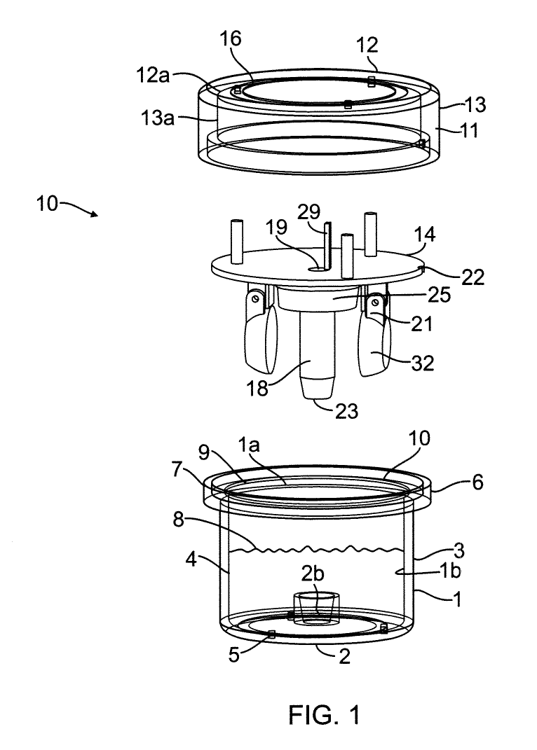

FIG. 1 shows an embodiment of a cleaning/disinfecting assembly 10 provided in accordance with the present invention. Although phrased as a “cleaning/disinfecting” assembly, the assembly is understood to be capable of providing one or the other or both functions. In one exemplary embodiment, the cleaning/disinfecting assembly 10 comprises a container or base 1 and a cap 11. The container or base 1 can be made from any suitable natural, synthetic, or laminate materials and most preferably from a transparent or translucent material. In one embodiment, the container 1 comprises a bottom wall 2 and a side wall 3 defining a container cavity 1a having an opening 7. Although shown with a single cylindrical side wall 3, the container may take on any appropriate shape and configuration and have distinct wall panels, such as a square container with four side wall panels.

In one embodiment, the bottom wall 2 comprises an anchor 2b and vents 5 that serve to relieve pressure build up inside the container 1 during the disinfection process, as further described below. The anchor 2b may embody an integrated cylinder having a tapered internal bore for engaging a stem, as further discussed below. As shown, the vents 5 are hollow bosses having hydrophobic filters that can be made from a number of suitable materials well known to one skilled in the art, such as super hydrophobic polyvinyldiflouride (PVDF). The vents allow gas to escape but not liquid. Other vents are contemplated, such as various other relief valves. The side wall 3 comprises a peripheral flange or mating rim 6, and comprises external threads 9. The peripheral flange 6 and external threads 9 are adapted to receive the cap 11 to seal the container 1, as will be further described in connection with the cap 11. The side wall 3 further comprises a fill line or demarcation 8 to indicate the level to which a disinfectant agent should be filled during use.

Within the container cavity 1a, an internal wall 4 having an internal opening 10 for an internal cavity 1b is provided. The internal opening 10 is recessed from the opening 7 located at the peripheral flange 6 and the internal cavity 1b is a subset of cavity 1a, located generally below the peripheral flange.

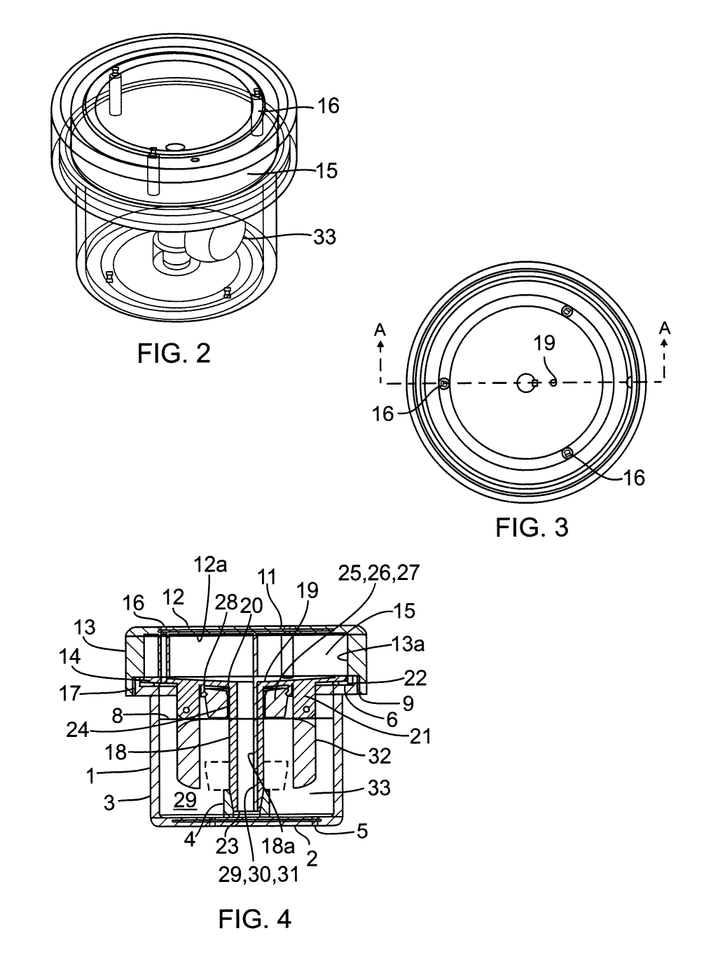

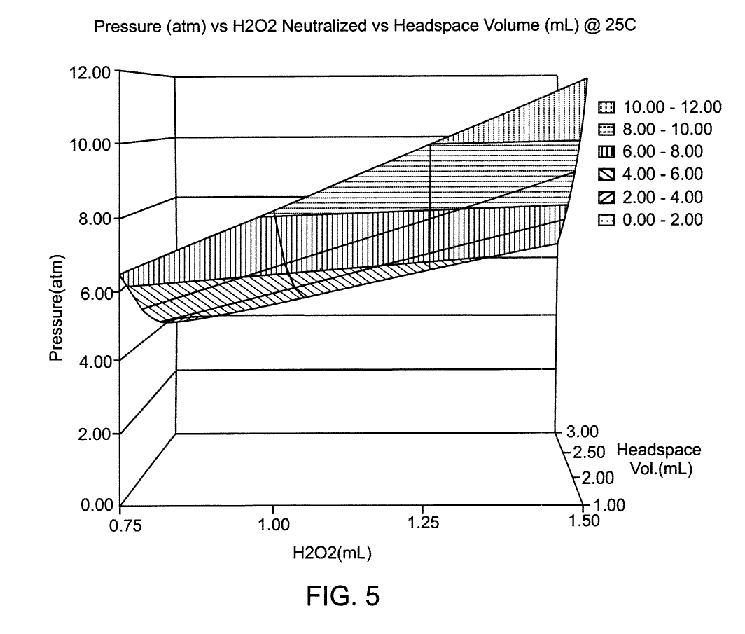

As shown in FIGS. 2 and 4, the closing of the cap 11 onto the container 1 divides the cleaning/disinfecting apparatus according to aspect of the present invention into 2 chambers, for instance a lower or first chamber 33 and an upper or second chamber 15. The designations first and second chambers serve to distinct the two chambers from one another but otherwise do not have structural limitations. The first chamber 33 may be referred to also as a disinfecting chamber and the second chamber 15 may be referred to as a pressure chamber. The two chambers are in communication with each other partly via vent holes, for example vent hole 19. Although FIGS. 2 and 4 show the two chambers stacked vertically one on top of the other, it should be understood that the other configurations could also be contemplated without departing from the scope of the invention. For instance, the chambers can be connected horizontally or in alternative configurations.

The cap 11 comprises a cap top wall 12, a cap side wall 13 and a cap bottom wall 14. As shown in FIGS. 2 and 4, the cap bottom wall 14, an internal cap side wall 13a and an internal cap top wall 12a define the pressure chamber 15. The cap side wall 13 comprises internal threads 17 configured to engage with the external threads 9 from the container 1 to mediate the sealing of the cap 11 with the container 1.

According to one embodiment of the invention, a first chamber or lower chamber 33 is defined by the cap bottom wall 14, the internal side wall 4 and the bottom wall 2. Within the first or lower chamber 33, lens baskets or holders 32 are attached to the bottom wall 14 via attachment means 21, which may comprise any suitable mechanical attachment fixtures such as pin, detents, etc. A hollow tube, projection, or stem 18 comprising a bore in fluid communication with the upper chamber extends distally from the bottom wall 14 and comprises an opening 23. The stem 18 is configured to project into and engage with the anchor 2b located at the bottom wall 2 of the container 1 upon sealing of the container 2 by the cap 11. The stem has a tapered tip configured to mate with the tapered internal bore of the anchor in a tapered fit arrangement.

A first neutralizing agent, such as a primary neutralizing component 25, is attached to the bottom wall 14 by sliding it up around the outside of the stem 18 and using friction, detent engagement, or a temporary fastener to hold in place. In one embodiment, the primary neutralizing component 25 comprises a support material 26 covered with a platinum coating 27, and one or more male interference closure seat 28 for engaging with female interference seat 20 located on the stem 18 to secure the primary neutralizing component 25 in place. The reverse detent arrangement between the neutralizing component 25 and the cap is also contemplated. The component 25 resembles a doughnut and has annular recess around the external periphery to serve as a female detent. As such, the component 25 has an outside diameter and an inside diameter.

A second agent, such as a secondary neutralizing component 29, is incorporated and held within a cavity 18a defined by the hollow interior of the stem 18. The secondary component 29 may comprise a support material 30 covered with a platinum coating 31 and having a male interference closure seat 31 for attaching to either the stem and/or the cap. A color indicator 24 extends proximally from an opening 19 on the bottom wall 14 and is in communication with the cavity 18a for providing user feedback information, as further discussed below.

Vents 19, 22 are provided on the bottom wall 14 of the cap to relieve pressure build up inside the first chamber 15 during the disinfection process, as further described below. Upper vents 16 are also provided but are provided with tubes that are connected to the lower cap wall 14. The vents 16 comprise cylindrical bosses that are each covered with hydrophobic material such as super hydrophobic polyvinyldiflouride (PVDF). Pressure build up in the lower chamber 33 can thus vent through the upper vents 16, through the tubes, and the lower vents 5 at the bottom of the container. The vent hole or opening 22 at the bottom cap wall is provided at a corner of the bottom wall 14 to allow gas release into the lower chamber to allow hydrogen peroxide to be captured in the stem upon closure of the cap onto the container, as will be further described. Vent 19 is configured as a relief valve by allowing pressure build up in the upper chamber to push against the primary neutralizing agent 25 to separate the same from the cap, as further discussed below. By providing vent holes 16 on the cap and vent holes 5 on the lower container, the apparatus can operate, i.e., can vent, whether it is held in the standing position shown in FIG. 2, flipped upside down, or tipped on its side.

Mode of Operation:

As shown in FIG. 4, in one exemplary embodiment, to use the cleaning/disinfecting assembly in accordance with aspects of the present invention, a cleaning/disinfecting agent, for example, hydrogen peroxide solution is placed into container 1, up to the fill line or demarcation line 8. Item(s) to be disinfected, for example contact lenses, are placed into holder(s) or basket(s) 32, which is well known in the prior art and has a plurality of strainer holes.

The cap 11 is then sealed to the container 1 by threading the cap 11 internal threads 17 onto the external threads 9 of the container. During the sealing process, liquid enters the hollow stem 18 through the opening 23 at the stern. This is possible because the displacement volume of gas in the pressure chamber 15 is expelled through the opening 22 near the peripheral edge of the lower cap wall 14. Also during the sealing process, the exterior of the distal end wall of stem 18 forms an air tight seal with internal wall 4, more particularly with the anchor 2b located on the bottom wall of the container.

FIG. 1 shows a cut-out on only one edge of the lower cap wall 14 with multiple cut-outs contemplated to provide additional vent openings. Upon full closure, opening 22 is air-tight sealed by the mating of the distal portion of the cap 11, i.e., the bottom wall 14, with the mating rim or peripheral flange 6 of the container. As such, the upper pressure chamber 15 is defined by the top wall 12, the side wall 13, and the bottom wall 14 (including the hollow of stem 18). The upper chamber is also defined by the two mating air tight seals formed upon closure by the distal end of stem 18 to the anchor at the internal wall 4 and the bottom wall 14 against the mating rim 6.

Upon closure of the cap to the container, the quantity of liquid hydrogen peroxide isolated within the hollow space of the stem 18 comes into communication with the secondary neutralizing component 29. The reaction of the hydrogen peroxide with the secondary neutralizing component 29 results in gas formation. Factors, among others, such as the area of the interface therebetween, the amount of hydrogen peroxide trapped by the stem, the volume of the pressure chamber 15, and the activity of coating 31 can contribute to the rate of gas formation. The rate can be controlled by manipulating these factors so that the pressure chamber 15 is pressurized to a desired pressure point after a desired set time frame. As it is formed, the pressure applies a force onto the engagement between the detents 20 and 28 which secures the primary neutralizing component 25 to the bottom wall 14. After a given time, at which point sufficient force has been achieved, the neutralizing component 25 is dislodged from its attachment to the bottom wall 14 and drops into the disinfecting chamber 33, which then opens a seal or channel 19 located between the two chambers that was previously sealed by the neutralizing agent 25. The detachment can also be regulated by the extent of physical engagement, such as depth of engagement and strength of material used, between the two detents 20, 28. The physical contact of the neutralizing component 25 with the liquid hydrogen peroxide initiates the neutralization reaction within the disinfecting chamber 33, which results in the formation of gas bubbles.

Graph 1 shows the relationship between the secondary neutralizing component 29 for a given volume of 3% hydrogen peroxide and for a given volume of head space in the upper pressure chamber 15. The information allows a skilled artisan to select the force or pressure necessary for a given head space to separate the primary neutralizing component 25 from the cap so that it falls into the bath of hydrogen peroxide to neutralize the disinfecting chamber 33. Again, so as to avoid pressure induced leakage or compromised physical integrity of the apparatus, a plurality of vents 5 and 16 are present to relieve the pressure formed in disinfecting chamber 33. Among other factors, the shape and size of the primary neutralizing component 25 and its location relative to basket(s) 32 can be designed and controlled in such a way as to generate sufficient heat as well as agitation to optimize the cleaning of the articles for use in basket(s) 32.

When the primary neutralizing component 25 is dislodged from its attachment to the stem 18 and the bottom wall 14, the color indicator band 24 which is hidden until then, becomes exposed. Its exposure thus indicates to the user that the neutralizing reaction has started and the contact lens will soon be ready for use after a specified period, for instance after a few hours, such as 4 to 6 hours, and/or upon confirmation that bubbles are no longer formed by the reaction.

In one embodiment, the lens basket(s) 32 are oriented with a central bias such that when the cap 11 forms a closure with the container 1, the percent fill volume to total internal volume is such that the lenses in the baskets stay submerged regardless of the apparatus's orientation.

Although limited embodiments of the cleaning/disinfecting assembly and its components have been specifically described and illustrated herein, many modifications and variations will be apparent to those skilled in the art. For example, various types of vents could be used, the attachment of the primary neutralizing component can be mediated by different engagement means such as friction fitted closure, such as a pressure fit closure, or a snap-fit closure, or a pop-off closure or the like; the seal between the cap and the container can be strengthened with the use of gaskets or O-rings or the like. Alternatively, metals other than platinum could be used to coat the support for the neutralizing components, or other hydrogen peroxide neutralization catalysts such as enzymes like catalase could be used, or non-catalytic neutralizers such as pyruvate and the like could also be used. Furthermore, the neutralizing components could be in alternative forms other than supported solids, such as tablet or powder forms. Additionally, although the above describes an improvement where the neutralizer is a supported neutralizer in communication with the chamber containing the item(s) to be disinfected, it should be obvious to one skilled in the art that any reasonable form of the aforementioned neutralizers could be isolated from that chamber and the pressure could be utilized to force open a release mechanism that only subsequent to sufficient pressure and release puts the neutralizer in communication with the chamber containing the item(s) to be disinfected.

Many alterations and modifications may be made by those having ordinary skill in the art, without departing from the spirit and scope of the invention. Therefore, it must be understood that the illustrated embodiments have been set forth only for the purposes of examples, and that the embodiments should not be taken as limiting the invention as defined by the following claims. The following claims are, therefore, to be read to include not only the combination of elements which are literally set forth, but all equivalent elements for performing substantially the same function in substantially the same way to obtain substantially the same result. The claims are thus to be understood to include those that have been illustrated and described above, those that are conceptually equivalent, and those that incorporate the ideas of the invention.

What is claimed is:

1. An apparatus adapted to delay the release of a primary agent, the apparatus comprising:
a first chamber fillable with a disinfectant agent and comprising a housing comprising a side wall, a top wall, a projection extending away from an internal housing wall and having a hollow bore, a secondary agent disposed, at least in part, in the hollow bore of the projection; wherein the primary agent being removably secured to the projection and covers an opening in the internal cap wall;
a second chamber comprising a housing of larger volume than the first chamber having multiple vent holes and in contact with the projection;
wherein the primary agent is displaceable from the projection by pressure generated in the first chamber after a time period measured from when the housing of the first chamber is coupled to the housing of the second chamber.

2. An apparatus for the timely controllable cleaning and disinfecting of articles for use, the apparatus comprising:
a first chamber having a first volume;
a second chamber having a second volume;
a seal isolating the first chamber from the second chamber;
a stem projecting from the first chamber into the second chamber; and
wherein the seal is severable by pressure build up in the first chamber; and
wherein a channel is created and linking the first chamber with the second chamber when the seal is severed.

3. The apparatus of claim 1, wherein the seal is a neutralizing agent having an outside diameter and an inside diameter.

4. The apparatus of claim 3, further comprising a neutralizing agent located, at least in part, within a hollow bore of the stem.

5. A method for delaying the release of a primary agent during the cleaning and disinfecting of articles for use, the method comprising:
adding a disinfectant agent into a container to a filled level;
placing an article to be disinfected into a holder;
placing a cap over the container and so that at least a portion of the holder is below the filled level of the disinfectant agent;
maintaining a neutralizing agent below the cap but above the filled level of the disinfectant agent;
moving the neutralizing agent into the disinfectant agent after a period of time in which pressure build up inside the cap and container is greater than atmospheric pressure.

6. The method of claim 5, further comprising an interior wall separating the cap and the container into an upper chamber and a lower chamber.

7. The method of claim 6, wherein the pressure build up is maintained in the upper chamber so that pressure in the upper chamber is greater than the lower chamber, for at least a finite length of time.

8. The method of claim 6, wherein the neutralizing agent temporary seals a channel formed on the interior wall separating the cap and the container.
